(19) United States
(12) Patent Application Publication
(10) Pub. No.: US 2025/0160885 A1
(43) Pub. Date: May 22, 2025

(54) ACCESS TOOL FOR DELIVERING CARDIAC THERAPIES TO THE PERICARDIAL SPACE

(71) Applicant: CHILDREN'S NATIONAL MEDICAL CENTER, Washington, DC (US)

(72) Inventors: Charles BERUL, Washington, DC (US); Justin OPFERMANN, Washington, DC (US); Rohan KUMTHEKAR, Washington, DC (US); Paige MASS, Washington, DC (US); Christopher SCHOLL, Washington, DC (US); Dylan PAPROSKI, Washington, DC (US)

(73) Assignee: CHILDREN'S NATIONAL MEDICAL CENTER, Washington, DC (US)

(21) Appl. No.: 18/835,259

(22) PCT Filed: Feb. 9, 2023
(86) PCT No.: PCT/US2023/012657

§ 371 Date: Aug. 1, 2024

Related U.S. Application Data
(60) Provisional application No. 63/308,224, filed on Feb. 9, 2022.

(51) Int. Cl. A61B 17/02; A61B 1/32

(52) U.S. Cl. CPC A61B 17/0218; A61B 1/32; A61B 2017/0225; A61B 2017/0237

(57) ABSTRACT

A surgical access port including an outer sheath having a tapered distal end; an inner sleeve fitting inside the outer sheath; and a cannulated core fitting inside the inner sleeve; wherein a distal opening of the outer sheath is configured to expand when the inner sleeve is inserted into the outer sheath, and wherein the cannulated core forms a first working channel and a second working channel between a first end of the cannulated core and an opposing second end of the cannulated core.

### CROSS-REFERENCE TO RELATED APPLICATIONS

The present application claims priority to U.S. Provisional Application No. 63/308,224, filed Feb. 9, 2022, which is incorporated herein by reference in its entirety for all purposes.

### FEDERAL FUNDING DISCLOSURE

This disclosure was made with government support under Grant Number 1R43HL144352-01 awarded by the National Institutes of Health. The government has certain rights to the disclosure.

### BACKGROUND

### FIELD OF THE DISCLOSURE

The present disclosure pertains to surgical ports enabling access to the pericardial space for endoscopy and delivery of cardiac therapies.

### DESCRIPTION OF THE RELATED ART

Cardiac therapies can be delivered to the heart via percutaneous or open surgical methods. There is a need for a minimally invasive approach to accessing the pericardial space and the heart while enabling direct visualization of delivery procedures.

The foregoing “Background” description is for the purpose of generally presenting the context of the disclosure. Work of the inventors, to the extent it is described in this background section, as well as aspects of the description which may not otherwise qualify as prior art at the time of filing, are neither expressly nor impliedly admitted as prior art against the present disclosure.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a surgical access port, comprising an outer sheath having a tapered distal end; an inner sleeve fitting inside the outer sheath; and a cannulated core fitting inside the inner sleeve; wherein a distal opening of the outer sheath is configured to expand when the inner sleeve is inserted into the outer sheath, and wherein the cannulated core forms a first working channel and a second working channel between a first end of the cannulated core and an opposing second end of the cannulated core.

In one embodiment, the present disclosure is related to surgical access port, comprising an outer sheath having a tapered distal end; an inner sleeve fitting inside the outer sheath; and a cannulated core fitting inside the inner sleeve; wherein a distal opening of the outer sheath is configured to expand when the inner sleeve is inserted into the outer sheath, wherein the cannulated core forms a first working channel and a second working channel between a first end of the cannulated core and an opposing second end of the cannulated core, and wherein the first working channel and the second working channel form an angle of separation.

In one embodiment, the present disclosure is related to a surgical access port, comprising an outer sheath having a tapered distal end; an inner sleeve fitting inside the outer sheath; and a cannulated core fitting inside the inner sleeve; wherein a distal opening of the outer sheath is configured to expand when the inner sleeve is inserted into the outer sheath, wherein the inner sleeve is removably coupled to the outer sheath, wherein the cannulated core forms a first working channel and a second working channel between a first end of the cannulated core and an opposing second end of the cannulated core, wherein the first working channel and the second working channel form an angle of separation, and wherein an outer wall of the cannulated core forms a notch at a proximal end of the cannulated core oriented towards a proximal opening of the second working channel.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an exploded view of a surgical access port, according to one embodiment of the present disclosure;

FIG. 2A is a front view of an outer sheath of a surgical access port, according to one embodiment of the present disclosure;

FIG. 2B is a lateral view of an outer sheath of a surgical access port, according to one embodiment of the present disclosure;

FIG. 3A is a front view of an inner sleeve of a surgical access port, according to one embodiment of the present disclosure;

FIG. 3B is a lateral view of an inner sleeve of a surgical access port, according to one embodiment of the present disclosure;

FIG. 4A is a front view of a cannulated core of a surgical access port, according to one embodiment of the present disclosure;

FIG. 4B is a lateral view of a cannulated core of a surgical access port, according to one embodiment of the present disclosure;

FIG. 5A is an assembled surgical access port in a retracted position, according to one embodiment of the present disclosure;

FIG. 5B is front view of an assembled surgical access port in a retracted position, according to one embodiment of the present disclosure;

FIG. 5C is a lateral view of an assembled surgical access port in a retracted position, according to one embodiment of the present disclosure;

FIG. 5D is a top view of an assembled surgical access port in a retracted position, according to one embodiment of the present disclosure;

FIG. 5E is a bottom view of an assembled surgical access port in a retracted position, according to one embodiment of the present disclosure;

FIG. 6A is an assembled surgical access port in an exploded position, according to one embodiment of the present disclosure;

FIG. 6B is a front view of an assembled surgical access port in an exploded position, according to one embodiment of the present disclosure;

FIG. 6C is a lateral view of an assembled surgical access port in an exploded position, according to one embodiment of the present disclosure;

FIG. 6D is a top view of an assembled surgical access port in an exploded position, according to one embodiment of the present disclosure;

FIG. 6E is a bottom view of an assembled surgical access port in an exploded position, according to one embodiment of the present disclosure;

FIG. 7A is a method for assembly and usage of a surgical access port, according to one embodiment of the present disclosure;

FIG. 7B is a method for disassembly of a surgical access port, according to one embodiment of the present disclosure;

FIG. 8A is an illustration of an insertion of a surgical access port, according to one embodiment of the present disclosure;

FIG. 8B is an illustration of a usage of a surgical access port, according to one embodiment of the present disclosure; and

FIG. 8C is an illustration of a removal of a surgical access port, according to one embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

There are many conditions that necessitate the placement of a cardiovascular implantable electronic device (CIED) in the heart. CIEDs can include, for example, pacemakers and implantable cardioverter defibrillators, each of which include conductive leads that are placed on an exterior (epicardial) or interior (endocardial) surface of the heart to carry electric signals to the organ. CIEDs can be used to restore and/or maintain normal heart rate and rhythm or for cardiac resynchronization therapy (CRT) in patients with heart failure. Cardiac leads can be placed on or inside the heart via open chest surgery, wherein the leads are inserted into the body through a sternal or thoracic incision and sewn or screwed onto the surface of the heart. Open procedures are associated with risks such as tissue damage, infection, intrathoracic adhesions, painful recovery, and other post-operative complications. In addition, open surgery can be especially traumatic for pediatric patients or others with existing health complications.

Percutaneous implantation of CIEDs is an alternative to open chest surgery that typically involves accessing the heart via the subclavian vein in order to attach leads to endocardial tissue without a thoracotomy or similar incision. However, venous access to the heart is not possible for neonates, infants, children, and some adults with congenital heart disease due to small vasculature, potential venous obstructions, and/or other anomalies. There is therefore a need to develop a minimally invasive approach for accessing the thoracic cavity and pericardial space in order to deliver cardiac therapies for patients with varying anatomies and cardiac conditions. In addition, an approach that incorporates direct visualization of the heart via endoscopy can improve the ease and effectiveness of the procedure while eliminating the need for fluoroscopy, which can have damaging radiation effects on the body.

In one embodiment, the present disclosure is directed towards a surgical access port providing access to structures inside the body. The surgical access port can also be referred to herein as a thoracic port and can provide access to the thoracic cavity and structures therein, including the pericardial space, the heart, and the great vessels. Alternative and additional areas of use for the surgical access port can be compatible with the present disclosure. In some embodiments, the surgical access port can be inserted into the thoracic cavity via a small incision in the inferior part of the sternum. In some embodiments, the thoracic port can be approximately 1 cm in diameter; thus, the incision can be smaller than the typical incision made for open chest surgery or larger ports. Advantageously, the surgical access port can be secured in the incision for endoscopic visualization of the thoracic cavity and delivery of cardiac therapies while minimizing exposure of internal organs to the open air. In one embodiment, the thoracic port can be configured for simultaneous insertion of one or more surgical instruments, including, but not limited to, a trocar, an imaging device (e.g., an endoscope), a guide wire, a needle, and other tools known to be used for delivering cardiac therapies while minimizing risk of clashing or crowding between the insertable tools. Advantageously, the surgical access port of the present disclosure can be a low-profile tool that can be easily operated by fewer medical professionals than are typically needed for cardiac procedures.

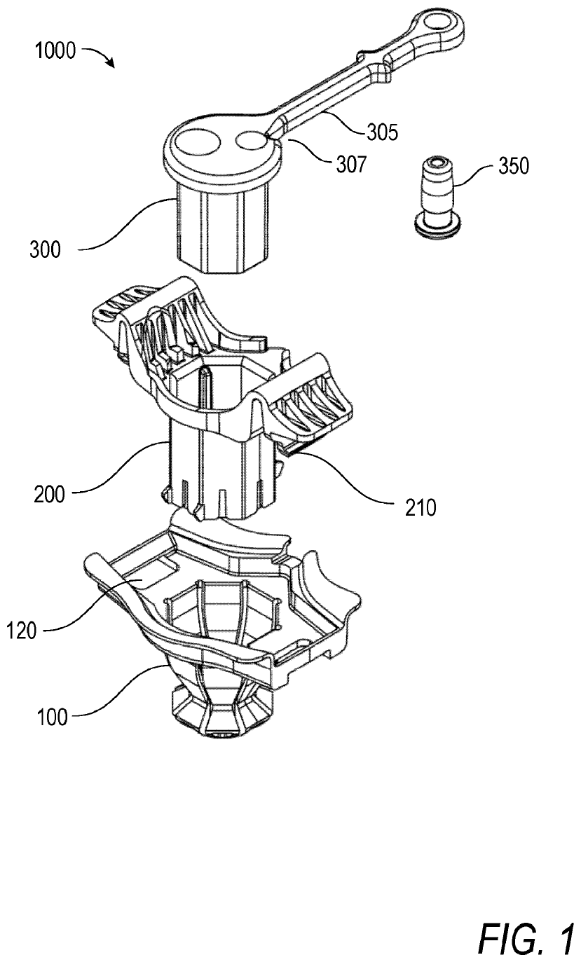

FIG. 1 is an exploded view of the thoracic port, according to one embodiment of the present disclosure. According to one embodiment, the thoracic port 1000 can include an outer sheath 100, an inner sleeve 200, and a cannulated core 300. A cannulated component can refer to a component with one or more channels running through the length of the component. The channel can terminate in an opening at each opposing end of the component. A cannula can be inserted through the channel to access the body. In one embodiment, the cannulated core 300, the inner sleeve 200, and the outer sheath 100 can each be approximately cylindrical in shape. The cannulated core can include one or more notches 307, which will be described in further detail herein. The outer sheath 100 can be hollow, with a proximal opening and a distal opening. The outer sheath 100 can form a chamber, wherein the inner sleeve 200 can be removably inserted into the chamber of the outer sheath. The inner sleeve 200 can also be hollow, with a proximal opening and a distal opening. The inner sleeve 200 can also form a chamber, wherein the cannulated core 300 can be removably inserted into the chamber of the inner sleeve. The distal end of the thoracic port 1000 can be inserted into a patient's body through an incision in the skin at or near the pericardial space. Surgical tools and instruments can be inserted through the proximal end of the thoracic port 1000 to reach the thoracic cavity.

In one embodiment, the distal end of the outer sheath 100 can be configured to secure the outer sheath 100 in the incision when the outer sheath 100 is inserted into the body, as will be described in further detail herein. In one embodiment, the outer sheath 100 can include locking structures 120, e.g., cavities, at the proximal end of the outer sheath. The locking structures 120 can couple with corresponding structures 210 at the proximal end of the inner sleeve 200 to temporarily affix the inner sleeve 200 inside the outer sheath 100. The cannulated core 300 can include a cannulated plug 350, wherein the plug 350 can be attached to the body of the core 300 via a tether 305. The cannulated core 300 can be inserted into the inner sleeve 200 and can be secured in the inner sleeve 200 by a combination of physical and material features.

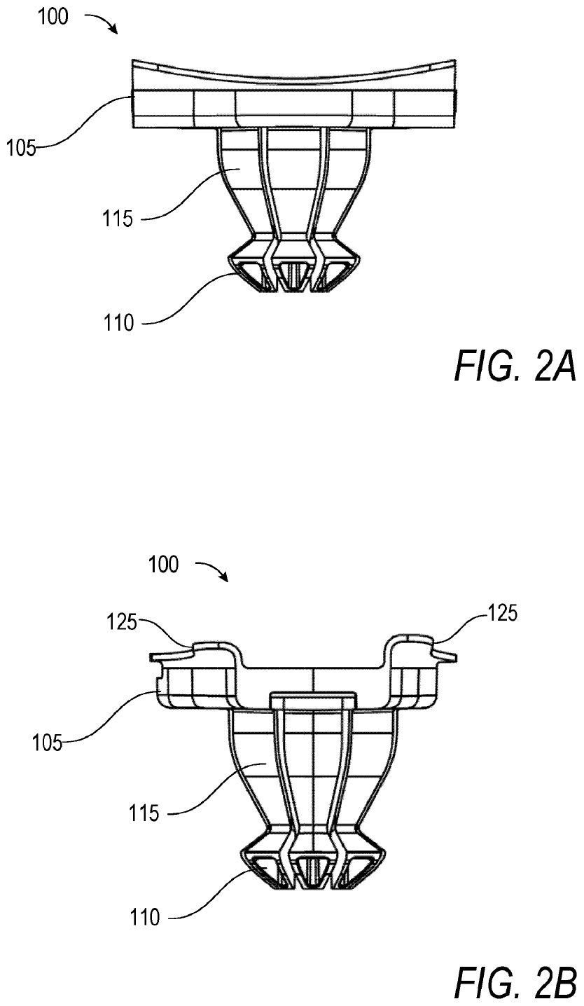

FIG. 2A is a front view of the outer sheath 100 in a retracted state, according to one embodiment of the present disclosure. The retracted state of the outer sheath 100 can be a default state when the outer sheath 100 is not inserted into the body and/or when the inner sleeve is not fully inserted into the outer sheath 100. The proximal end of the outer sheath 100 can include a circular opening and a flat shelf 105 surrounding the opening. The flat shelf 105 can sit on or above the skin when the outer sheath 100 is inserted into the body, providing a stopping point for insertion of the outer sheath 100. In one embodiment, the outer sheath 100 can include a tapered distal end in the retracted state, as illustrated in FIG. 2A. The tapered distal end can enable a less traumatic insertion of the outer sheath into an incision in the body. For example, the distal end can be narrower than the incision, and the body of the outer sheath 100 can gradually open or widen the incision as the outer sheath 100 is inserted. The outer sheath 100 can be formed by one or more vertical slats 115. The vertical slats 115 can be joined at the proximal end of the outer sheath 100 such that the proximal end forms a continuous circumference or outer edge. In one embodiment, the vertical slats 115 can be curved to form the tapered distal end of the outer sheath 100. For example, the vertical slats 115 can be convex and can converge at the distal end of the outer sheath. The vertical slats 115 can be deformable or flexible such that the curvature and/or concavity of the vertical slats 115 can change upon application of a force. For example, the vertical slats 115 can be expanded or flexed by the application of a force against the inner walls of the vertical slats 115 and can flare outwards (away) from the central axis of the outer sheath. The separation between each vertical slat enables the widening of the distal end and the distal opening of the outer sheath when the vertical slats 115 are flexed. The expansion of the vertical slats 115 will be described in further detail herein.

In one embodiment, the distal end of each vertical slat 115 can include a projection forming a flange 110, the flange 110 projecting outwards (away) from the central axis of the outer sheath and forming a widened rim at the distal end. The flange 110 can anchor the outer sheath 100 in the incision when the outer sheath is inserted into the body and expanded. For example, when the vertical slats 115 are expanded, the flanges can be in contact with the inner wall of the thoracic cavity where the outer sheath is inserted. The expansion of the flanges will be described in further detail herein. In one embodiment, the flanges 110 can have flat or rounded edges so as to reduce irritation to the body. In one embodiment, the flanges 110 can be coated with a biocompatible silicon or similar semi-soft material to reduce irritation to the body.

FIG. 2B is a lateral view of the outer sheath 100, according to one embodiment of the present disclosure. In one embodiment, the proximal shelf 105 of the outer sheath 100 can form one or more locking structures. In one embodiment, the locking structure can include the divot or cavity 120 in the upper surface of the proximal shelf, as illustrated in FIG. 1. A compatible structure of the inner sleeve can be inserted into the cavity to lock the inner sleeve to the outer sheath. In some embodiments, the proximal shelf 105 and/or the locking structure can be deformable. A force applied to the proximal shelf 105 can deform the cavity or another part of the shelf 105 to allow for coupling and decoupling of the inner sleeve. For example, the locking structure 120 can include one or more wings 125 extending from the sides of the proximal shelf, as illustrated in FIG. 2B. The wings 125 can be disposed on opposite sides of the proximal opening in the outer sheath. A force can be applied to the wings 125 to deform the proximal shelf, e.g., widen the cavity in a dimension perpendicular or parallel to the direction of the force. The widening of the cavity can enable the uncoupling of the structure of the inner sleeve that has been inserted into the cavity and the removal of the inner sleeve from the outer sheath.

In one embodiment, the thoracic port can be a low-profile port. The port can provide access to the thoracic space without extending deep into the body. In one embodiment, the body of the outer sheath 100 can be shorter than typical trocars used to access the thoracic space. The distal flanges of the outer sheath 100 can anchor the thoracic port inside the body even with the shorter length of the thoracic port. The length of the thoracic port is advantageous in that the shallow but secure insertion of the port reduces the risk of the port crossing the diaphragm or causing injury to the heart or lungs upon insertion. In one embodiment, the outer sheath 100 can be a medical-grade, biocompatible material. As an example, the outer sheath can be a styrene material, such as medical-grade acrylonitrile butadiene styrene (ABS) or commercial analog or equivalent. In one example, the outer sheath can be a plastic such as medical polyurethane or multi-purpose polyurethane (MPU), or any known analog or equivalent such as MPU100, a commercial product. In some embodiments, the outer sheath can be sterilizable and durable with a high tensile strength (e.g., approximately equal to or greater than 38 MPa (megapascal). In some embodiments, the outer sheath can be 3D printed or injection molded with engineering-grade mechanical properties.

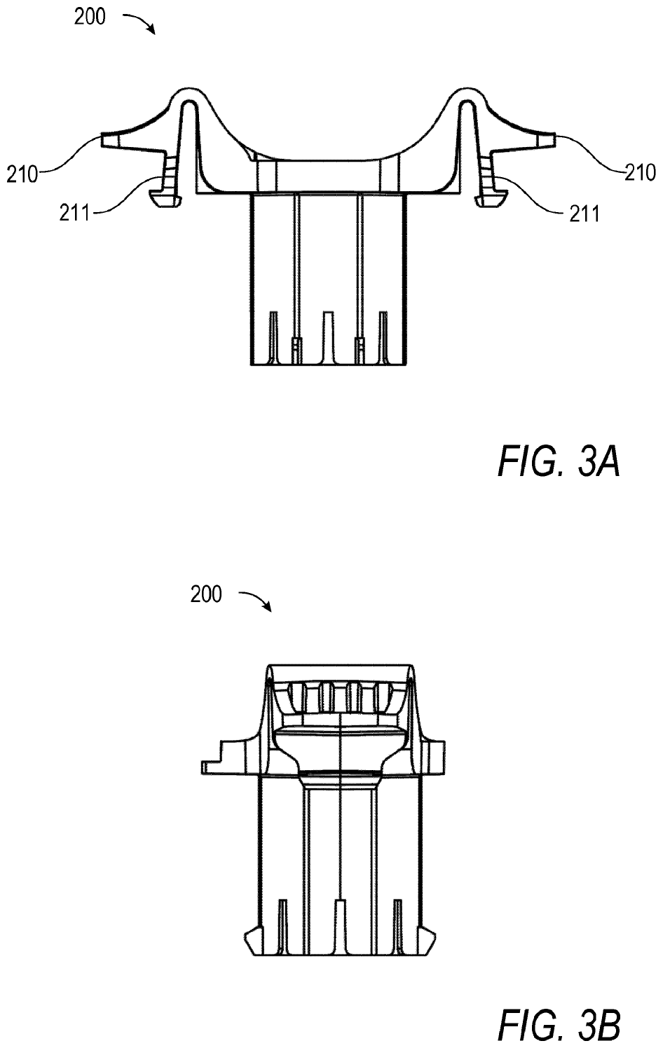

FIG. 3A is a front view of the inner sleeve 200, according to one embodiment of the present disclosure. The inner sleeve 200 can be approximately cylindrical. In some embodiments, the inner sleeve 200 can also be tapered. The proximal end of the inner sleeve can include a circular opening and one or more clips 210 surrounding the circular opening and extending outwards from the circular opening. The inner sleeve can be inserted into the outer sheath until the bottom surface of the clips 210 is in contact with the upper surface of the proximal shelf of the outer sheath. The clips 210 can thus provide a stopping point for insertion of the inner sleeve. In some embodiments, the clips 210 can couple with the proximal shelf of the outer sheath in order to attach the inner sleeve to the outer sheath. For example, the bottom surface of the clips can form one or more slats or projections 211 extending downwards. In one embodiment, the projection 211 can extend across a dimension (e.g., a width) of the inner sleeve. The projection 211 can terminate in a widened base. The projection 211 can be inserted into the cavity in the upper surface of the outer sheath to couple the inner sleeve 200 to the outer sheath.

FIG. 3B is a lateral view of the inner sleeve 200, according to one embodiment of the present disclosure. In some embodiments, the distal end of the inner sleeve can include one or more raised structures, such as bumps, ridges, or projections on the outer surface of the inner sleeve. The raised structures can provide additional force on or displacement of the slats of the outer sheath when the inner sleeve is inserted into the outer sheath. In one embodiment, the distal end of the inner sleeve can include one or more cutouts or slots. The slots can improve the deformability of the inner sleeve so as to prevent breakage, e.g., when the cannulated core is inserted into the inner sleeve or when a torsion force is applied.

In one embodiment, the inner sleeve 200 can be a biocompatible polymer material, such as polyurethane. In one embodiment, the inner sleeve can be a rigid polyurethane (RPU) such as RPU70 or a similar or equivalent analog. In one embodiment, the tensile strength of the inner sleeve can be approximately equal to or greater than that of the outer sheath (e.g., 40 MPa). The outer sheath and the inner sleeve can be comparatively stiffer and/or stronger than the cannulated core of the thoracic port. In some embodiments, the inner sleeve can be sterilizable. In some embodiments, the inner sleeve can be 3D printed or injection molded with engineering-grade mechanical properties.

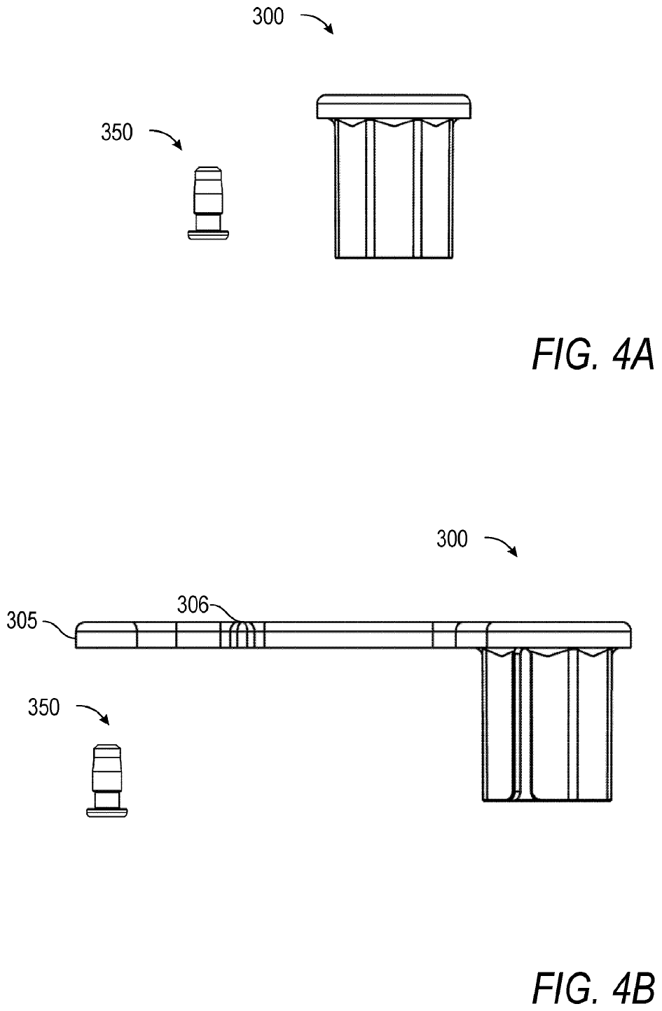

FIG. 4A is a front view of a cannulated core 300, according to one embodiment of the present disclosure. The cannulated core 300 can be a cylindrical body with one or more working channels through the cylindrical body. The one or more working channels can be used for advancement of surgical instruments and tools through the thoracic port when the cannulated core is inserted into the inner sleeve. In one embodiment, the proximal end of the cannulated core 300 can be wider than the body of the core. In some embodiments, the core can include a cannulated plug 350. The cannulated plug 350 can form at least one working channel through the length of the plug 350. The cannulated plug 350 can be inserted into a working channel of the cannulated core 300 in order to narrow the working channel for use with certain tools or devices (e.g., a needle or cardiac lead).

FIG. 4B is a lateral view of the cannulated core 300, according to one embodiment of the present disclosure. The cannulated plug 350 can be attached to the cannulated core 300 via a tether 305. The tether 305 can be a flexible material and can fold or bend so that the cannulated plug 350 can be inserted into the core 300. In one embodiment, the plug 350 can be removably attached to the end of the tether 305. For example, the plug 350 can be inserted into a hole in the end of the tether 305. In one embodiment, the outer sheath and the inner sleeve can each include a notch at the proximal end wherein the tether can be fitted into the notch when the cannulated core is inserted into the inner sleeve. The tether can 305 be secured in the notch so that it does not interfere with access to and through the thoracic port when the plug is not in use. In one embodiment, the outer surface of the cannulated plug 350 can include raised or recessed structures such as ridges, bumps, etc. The raised structures can create friction between the outer surface of the cannulated plug 350 and the inner surface of the working channel so that the plug 350 is held in place inside the working channel.

In one embodiment, the core 300 can be used as a reference to indicate an appropriate length for a cardiac procedure requiring access to the thoracic cavity. In some procedures, an incision into the thoracic cavity can typically be made a set distance from a known anatomical structure to enable access to the pericardial space. For example, an incision can be made approximately 13 mm below the xiphoid process, which can be felt at the base of the sternum. The tether 305 can include one or more reference markings, projections, or similar features along the length of the tether as a reference point for a known length. For example, the tether 305 can include a demarcation or reference feature 306 along the length of the tether 305. The demarcation can be a visual marking or a structural feature along the length of the tether. The distance between the end of the tether 305, where the cannulated plug 350 is provided, and the reference feature 306 can be a known length such as 13 mm that is used to determine where to make an incision for implantation. In some embodiments, the reference features can indicate more than one length. For example, the distance between the reference feature and the body of the cannulated core or the distance between a first reference feature and a second reference feature can be a second known length that is typically used in an implantation procedure, such as a typical incision length. During a procedure, a user can use the tether 305 to determine an incision point that is 13 mm away from the xiphoid process, thus eliminating the need for guesswork or additional measurement tools.

In one embodiment, the cannulated core 300 can be an elastic core. For example, the cannulated core 300 can be a flexible polymer material, such as ethylene vinyl acetate copolymer or a polyurethane elastomer such as EPU40. In one embodiment, the cannulated plug 350 can be a rigid polyurethane (e.g., RPU70) or a similar analog. In some embodiments, the cannulated core can be sterilizable. In some embodiments, the cannulated core can be 3D printed or injection molded with engineering-grade mechanical properties. The cannulated core 300 can be removably inserted into the inner sleeve to provide working channels for access to the thoracic cavity and structures therein. In some embodiments, the cannulated core 300 can be removed from the inner sleeve to provide a wider access port to the thoracic cavity through the opening of the inner sleeve.

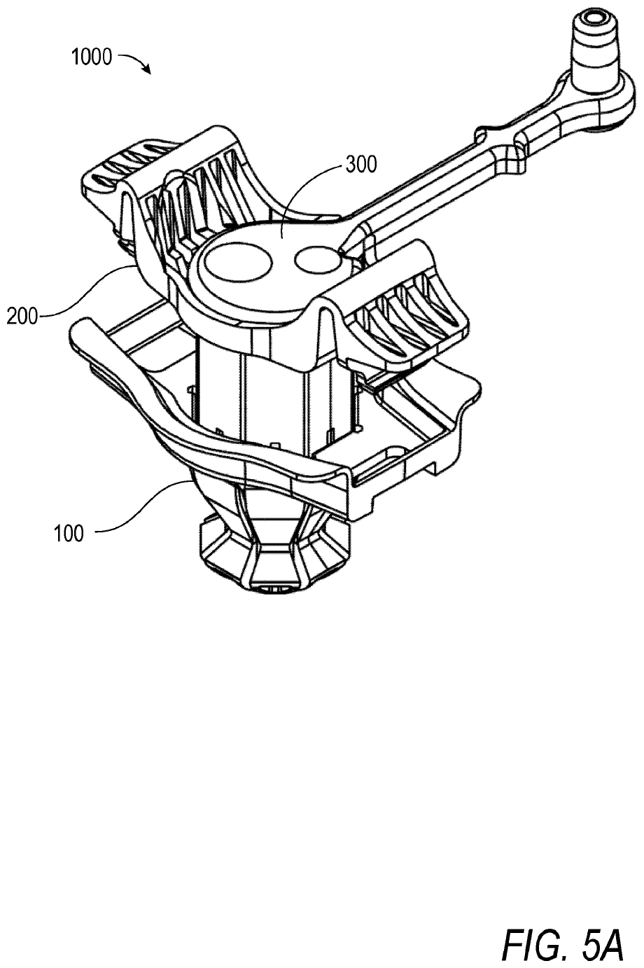

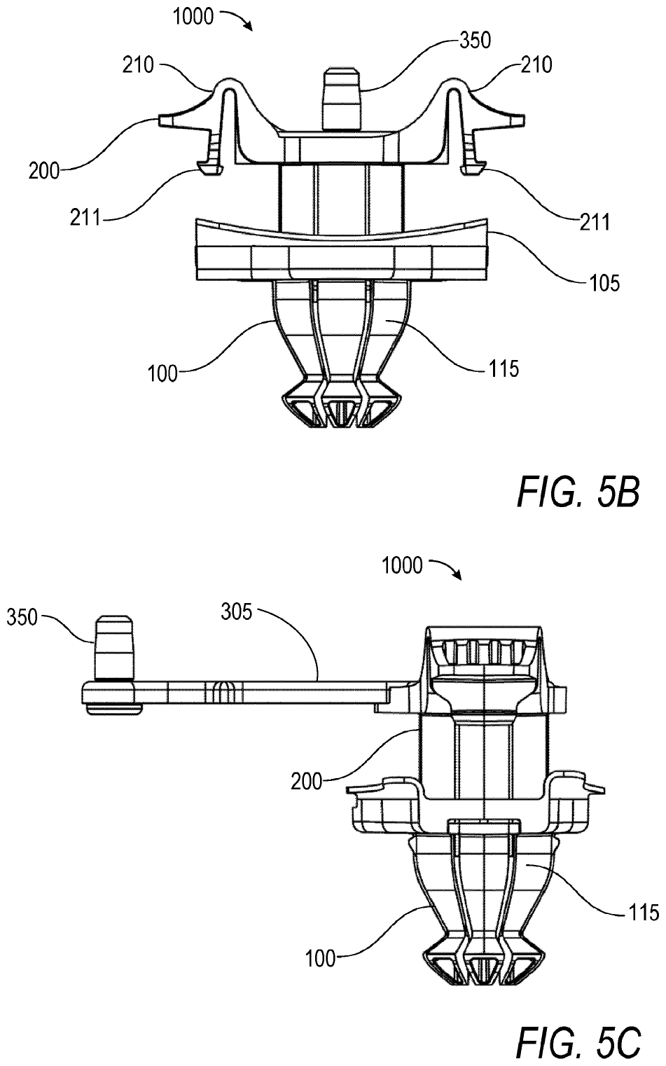

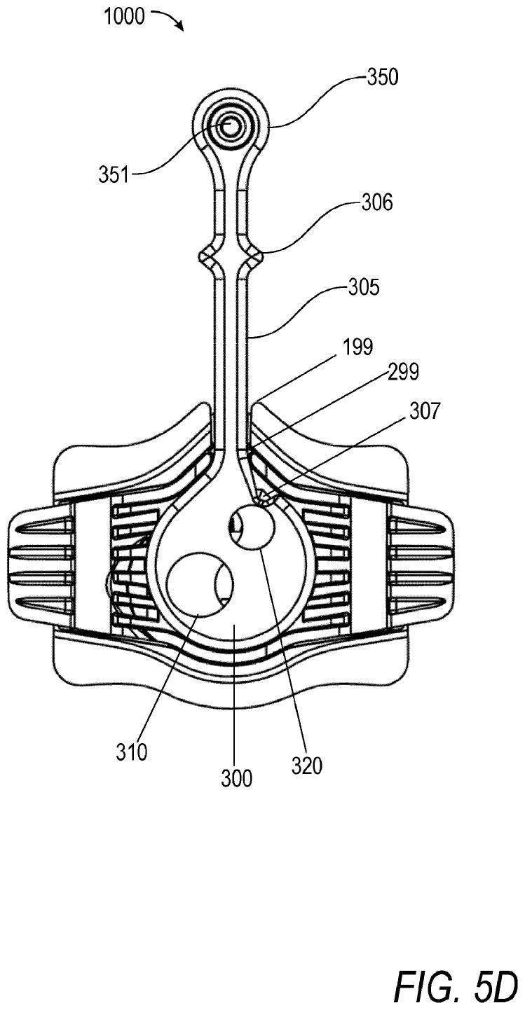

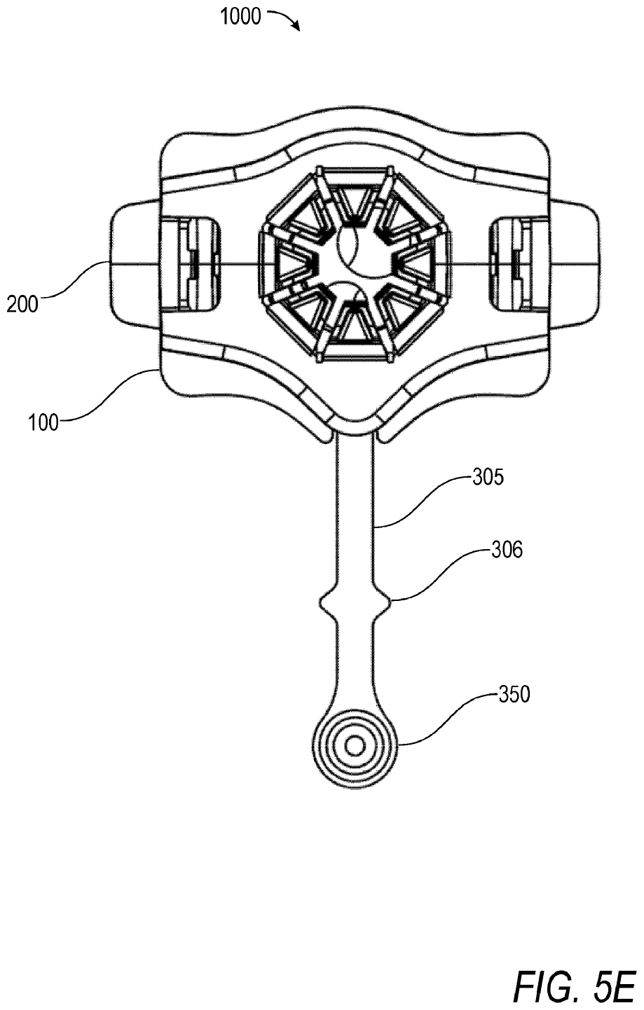

FIG. 5A is an illustration of the assembled thoracic port 1000 in a retracted state, according to one embodiment of the present disclosure. The cannulated core 300 can be inserted into the inner sleeve 200. The dimensions of the cannulated core 300 and the inner sleeve 200 can create a tight fit between the components such that a pushing or pulling force is needed to insert and remove the cannulated core 300. In one embodiment, the outer surface of the cannulated core 300 can include raised or recessed structures such as ridges, bumps, etc. The raised structures can create friction between the outer surface of the cannulated core 300 and the inner surface of the inner sleeve 200 so that the core 300 is held in place inside the inner sleeve 200. In the retracted state, the inner sleeve 200 can be partially inserted into the outer sheath 100 so that the proximal end of the inner sleeve 200 is not in line with the proximal end of the outer sheath 100. Accordingly, there can be space between the proximal end of the inner sleeve 200 and the proximal end (shelf) of the outer sheath 100 in the retracted state. In one embodiment, the inner sleeve 200 can remain partially inserted in the outer sheath 100 until a force is applied to push the inner sleeve 200 further into the outer sheath 100 and engage the outer sheath 100 in an expanded or flexed state. In one embodiment, the thoracic port can be partially assembled or can be assembled in a retracted state before the outer sheath 100 is inserted into an incision in the body. The inner sleeve 200 can then be fully inserted into the outer sheath 100 after the tapered distal end of the outer sheath 100 has been inserted into the incision.

FIG. 5B is a front view of the thoracic port 1000 in a retracted state, according to one embodiment of the present disclosure. The cannulated plug 350 can extend upwards from the tether attached to the core that is inserted into the inner sleeve 200. The projections 211 on opposite sides of the proximal opening of the inner sleeve 200 can be vertically aligned with the proximal shelf 105 of the outer sheath 100. In some embodiments, the clips 210 around the proximal opening of the inner sleeve 200 can extend past the edge of the outer sheath 100 in one or more directions. The vertical slats 115 of the outer sheath 100 can remain convex in the retracted state.

FIG. 5C is a lateral view of the thoracic port 1000 in a retracted state, according to one embodiment of the present disclosure. In the retracted state, the cannulated core can be fully inserted and contained in the inner sleeve 200. The tether 305 can extend outwards from the body of the inner sleeve 200. The inner sleeve 200 and the cannulated core are not fully inserted into the outer sheath 100. The vertical slats 115 of the outer sheath 100 are curved inwards.

FIG. 5D is an overhead view of the thoracic port 1000 in a retracted state, according to one embodiment of the present disclosure. In some embodiments, the cannulated core 300 can include a first working channel 310 and a second working channel 320 through the interior of the core 300. The working channels can be fully contained within the body of the core or can be contiguous with or tangential to the circumference of the core. In one embodiment, the first working channel 310 and the second working channel 320 can have different dimensions. In one example, the first working channel 310 can be a visualization channel and can be wider than the second working channel 320. A visualization channel can be, for example, approximately 5 mm in diameter, while the second working channel can be, for example, approximately 3.5 mm in diameter. The dimensions of the working channels can be configured such that the working channels can generate a drag force on an inserted instrument to prevent the instrument from slipping or being dislodged when inserted in the working channel.

The visualization channel 310 can accommodate a trocar for thoracic insufflation and an endoscope for thoracic imaging. In some embodiments, the insufflation trocar can be concentric with the endoscope. For example, an insufflation trocar can be inserted into the visualization channel 310 to inflate the thoracic cavity with a gas (e.g., carbon dioxide) for pericardial access. An endoscope, e.g., a deflectable endoscope, can be passed through the trocar and into the thoracic cavity. The endoscope can be adjusted to provide an operator with a live view of the heart and the thoracic cavity. The trocar and the endoscope can be secured in place at the insertion site by the thoracic port. In one example, the visualization channel can generate a drag force (e.g., 4N (Newtons)) on the inserted trocar such that the trocar does not become dislodged when the endoscope is inserted. The short profile of the thoracic port enables greater range of motion for the endoscope and any other instruments or devices inserted into the port. The second working channel 320 can be a working channel for insertion of a surgical instrument or device, including, but not limited to, a guide wire, a dilator, a sheath, a catheter, a cardiac lead, etc. The smaller diameter of the second working channel 320 can constrain lateral movement of an inserted instrument inside the channel so that an operator has more control over the instrument.

In one embodiment, the first working channel 310 and the second working channel 320 can be arranged within the cannulated core to reduce contact or interference within the thoracic cavity between a first instrument advanced past the distal end of the first working channel 310 and a second instrument advanced past the distal end of the second working channel 320. In one embodiment, the first working channel 310 and/or the second working channel 320 can be angled relative to the central axis of the cannulated core rather than parallel to the central axis. According to an exemplary implementation, the first working channel 310 can be angled relative to the second working channel 320 with an angle of separation of approximately 25°. The angle of separation between the first working channel and the second working channel can refer to an angle that would be formed by the channels if the channels were coplanar. Alternative angles of separation are compatible with the present disclosure. For example, the angle of separation can be greater than 25° or less than 25°. In one embodiment, the angle of separation can be an acute angle. In one example, the first working channel 310 and the second working channel 320 can be angled towards each other at the distal end of the core but can be offset (e.g., not coplanar) such that the inserted instruments do not cross.

The angled working channels can guide the advancement of the inserted instruments to prevent crossing, entanglement, intersection, or other interference at the distal end of the thoracic port. In some embodiments, the angled working channels can guide the inserted instruments to a region of the thoracic cavity (e.g., the heart) while maintaining the visualization of the inserted instruments. For example, a needle inserted into the second working channel 320 can be advanced towards the pericardial sac by following the angle of the second working channel. An endoscope inserted into the first working channel 310 can be advanced away from the needle, wherein the location of the endoscope in the thoracic cavity as a result of the angle of the first working channel 310 enables a field of view that includes the needle and the pericardial sac. The separation and angle of the working channels can optimize the surgical field such that all delivery tools remain visible in a captured endoscope image throughout the procedure.

In one embodiment, the outer wall of the cannulated core can form a notch 307 at a proximal end of the cannulated core oriented towards a proximal opening of the second working channel 320. The notch 307 can be a curved indentation as in FIG. 5D or can be pointed or angled. In one embodiment, the notch 307 can be approximately tangential to the proximal opening of the second working channel 320. The second working channel 320 can remain fully contained within the cannulated core 300. The notch 307 can indicate a location where the cannulated core 300 can be cut (e.g., with a scalpel) or torn to open the proximal opening of the second working channel 320. A cut at the notch 307 can connect the proximal opening of the second working channel to the outer wall of the cannulated core 300 such that the proximal opening can become a C-shaped opening rather than a circular opening. In some embodiments, a vertical cut can be made at the notch 307 to open a portion of the length of the second working channel 320 to become a C-shaped channel. In one embodiment, the outer wall of the cannulated core can form a seam or a perforated line that can be cut. The C-shaped opening and length of the second working channel 320 can enable the insertion and implantation of larger devices, such as a miniature pacemaker. In some embodiments, a device can be inserted into the body and implanted near the skin, e.g., at the inner wall of the thoracic cavity or in the incision wherein the port is inserted. The device can be inserted into the C-shaped second working channel 320. In one embodiment, the cannulated core can be removed from the inner sheath by maneuvering the C-shaped second working channel around the device that has been inserted therein rather than threading the entire device through the second working channel. The incision at the notch 307 can be a parting line for separating the cannulated core from an inserted device.

The cannulated plug 350 can be attached to the core 300 with the tether 305. The tether 305 can include one or more reference features 306 to indicate a known length of a portion of the tether. In one embodiment, the tether 305 can be secured in place at the inner and/or the outer sheath. As an example, the tether 305 can be fitted into the notch 199 of the outer sheath and the notch 299 of the inner sleeve, wherein the notch 299 of the inner sleeve sits inside or on top of the notch 199 of the outer sheath. In one embodiment, the tether 305 can remain in the notches when the cannulated plug 350 is inserted into one of the working channels in the core 300. The tether 305 can bend or fold at a point past the notch. The tether 305 thus does not interfere with access and operations through the thoracic port. In some implementations, the cannulated plug 350 can be inserted into a working channel that is not used for visualization, e.g., the second working channel 320 of FIG. 5D. The outer circumference of the cannulated plug 350 can be approximately the same as the inner circumference of the working channel. The cannulated plug 350 can form a working channel 351 through the body of the plug 350. The working channel 351 can be fully contained within the body of the plug 350 or can be contiguous with or tangential to the circumference of the plug 350. In one embodiment, the working channel 351 can be angled relative to the central axis of the plug 350. In one embodiment, the plug 350 can have an angled body (e.g., an oblique cylinder) or can be inserted into the working channel 320 at an angle to maintain the angle of the working channel 320. The working channel 351 can be narrower than the working channel 320. According to one example, the working channel 351 can be approximately 1.27 mm in diameter. The insertion of the plug 350 into the working channel 320 thus narrows the working channel 320 to approximately half of the diameter of the working channel 320. The narrower working channel 351 can be used for thin tools such as needles. The cannulated plug 350 can be approximately the length of the second working channel 320 or can be shorter than the second working channel 320. The insertion of the plug 350 does not interfere with the first working channel 310.

According to one use case, the plug 350 can be inserted into the second working channel 320 and a needle can be inserted through the working channel 351 and used to pierce the pericardial sac as a first step of cardiac therapy delivery. The needle can then be removed from the working channel 351 and the plug 350 can be removed from the second working channel 320. A larger instrument, such as a dilator, can then be inserted into the second working channel 320 to continue the procedure. In some embodiments, the cannulated plug 350 can prevent gas leakage from the thoracic cavity. For example, the thoracic cavity can be insufflated via a trocar inserted into the first working channel 310. If an instrument or device that is narrower than the second working channel 320 is inserted into the second working channel 320, the empty space between the instrument and the inner wall of the second working channel 320 provides a channel for gas to escape from the thoracic cavity. Gas leakage reduces the efficacy and efficiency of the insufflation. The plug 350 can fill the empty space in the second working channel 320 to prevent such leakage.

In some embodiments, the notches 199, 299 in the outer and inner sleeves can indicate the orientation of the thoracic port. For example, the thoracic port can be inserted into a subxiphoid process incision such that the notches and the tether of the cannulated core are oriented towards the head of the patient. The thoracic port can be oriented so that the angled working channels guide the inserted instruments towards designated regions in the thoracic cavity. For example, the proper orientation of the thoracic port in a subxiphoid process incision can ensure that the second working channel 320 is angled towards the heart, while the first working channel 310 is angled away from the heart. In one embodiment, the components of the thoracic port can include additional or alternative visual or structural features to indicate proper insertion orientation and usage.

In some embodiments, the thoracic port can be assembled without the cannulated core 300. For example, the thoracic port can provide access to the thoracic cavity for instruments that are larger than the working channels provided in the cannulated core. The outer sheath 100 can be inserted into an incision in the body and the inner sleeve 200 can be inserted into the outer sheath 100. The chamber formed by the inner sleeve 200 can be a surgical window, wherein instruments and devices can be inserted into the thoracic cavity directly through the surgical window. The diameter of the chamber formed by the inner sleeve 200 can be, in some embodiments, approximately 1 cm. The insertion through the inner sleeve 200 without the cannulated core 300 can be especially advantageous for delivering larger cardiac therapies. For example, the surgical window can be used to insert and attach patch epicardial leads and leadless pacemakers or to dissect pericardial adhesions in patients with prior cardiothoracic surgeries. The surgical window can provide direct visualization of the thoracic cavity through the window as well as a working channel for larger instruments.

FIG. 5E is a view of the thoracic port 1000 in a retracted state from the distal end of the port, according to one embodiment of the present disclosure. When the inner sleeve is partially inserted, the vertical slats of the outer sheath remain tapered at the distal end, as illustrated in FIG. 5E. In some embodiments, the tapered distal end of the outer sheath 100 can obscure or partially obscure the distal openings of the working channels in the cannulated core. The distal flanges of the outer sheath extend outwards from the central axis of the outer sheath. In one embodiment, the distal flanges can extend outwards from the central axis of the outer sheath and upwards towards the proximal end of the outer sheath. The angle of the distal flanges can increase contact between the distal flanges and the inner wall of the thoracic cavity when the port is in a flexed or expanded state.

The thoracic port can be expanded when in use in order to secure the outer sheath of the port to the body and lock the inner sleeve to the outer sheath. When the thoracic port is in an expanded state, each component can be secured in the incision and a user can focus on inserting and operating instruments through the port rather than holding the port in place. The thoracic port can be engaged in the expanded state when the inner sleeve is fully inserted into the outer sheath. The inner sleeve can be reversibly coupled to the outer sheath when fully inserted and can be locked in place. The thoracic port of the present disclosure can include a number of physical features and/or material properties to be secured to the body in the expanded state.

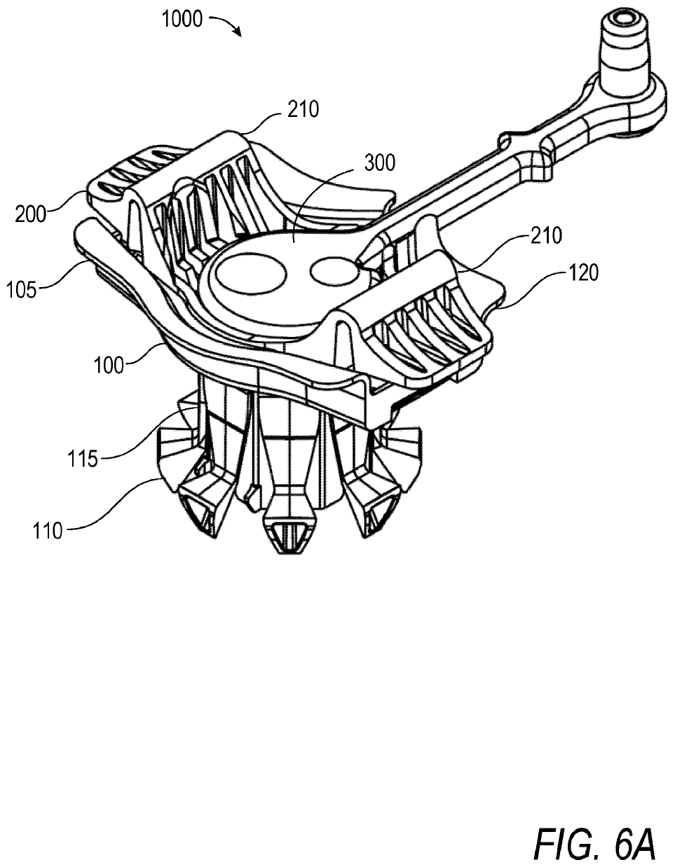

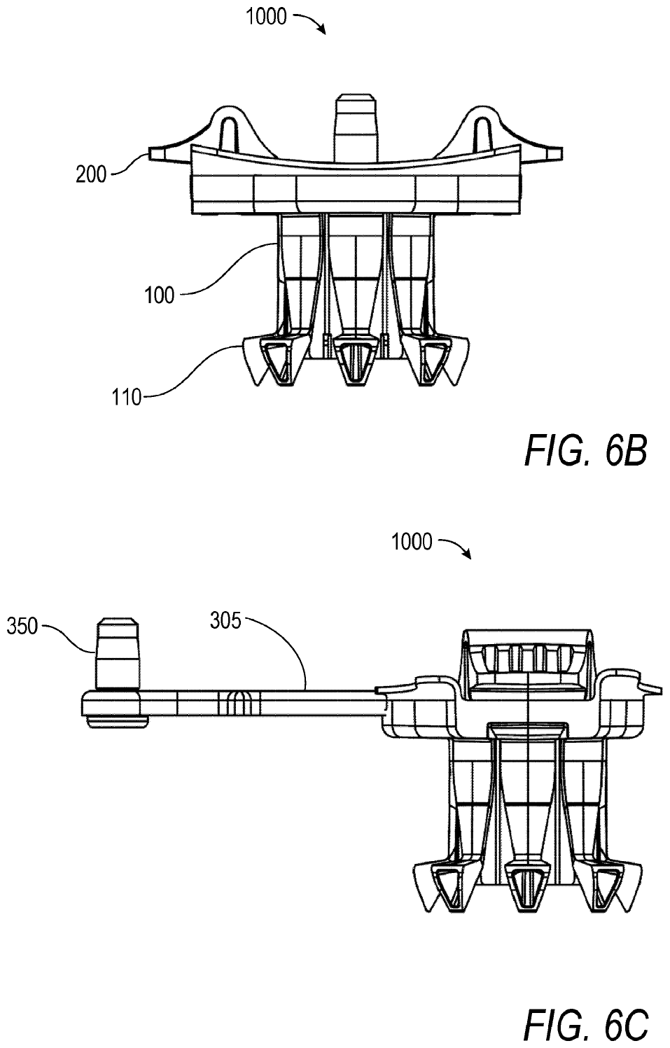

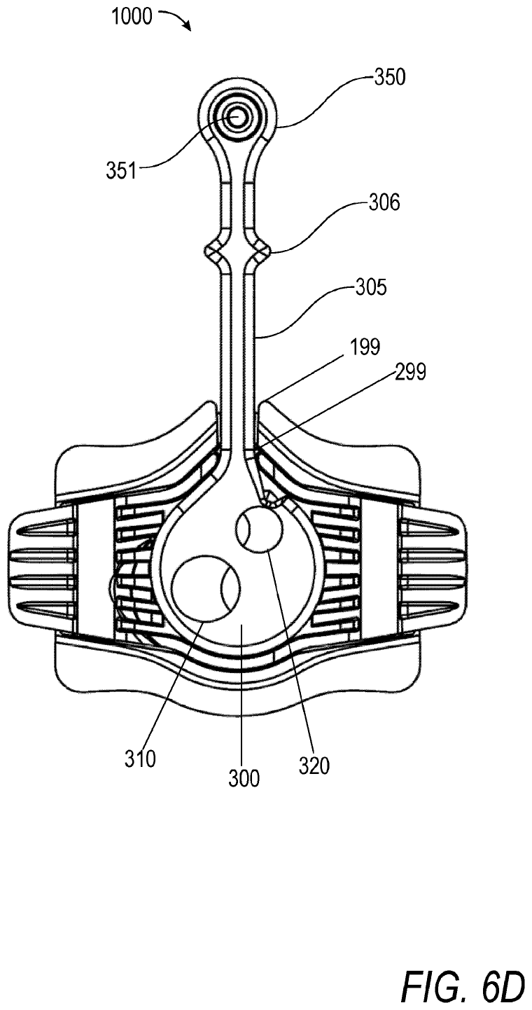

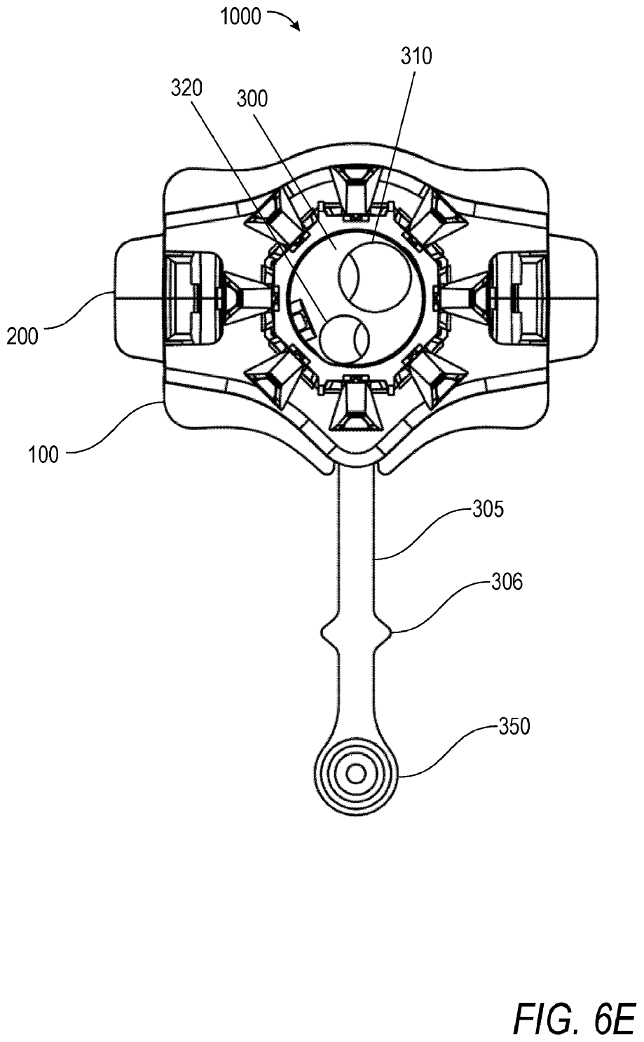

FIG. 6A is an illustration of the thoracic port 1000 in an expanded state, according to one embodiment of the present disclosure. In the expanded state, the inner sleeve 200 can be fully inserted into the outer sheath 100 such that the proximal opening of the inner sleeve is in line with the proximal opening of the outer sheath. According to one embodiment, the clips 210 on either side of the proximal opening of the inner sleeve 200 can be in contact with and/or coupled to the shelf 105 at the proximal end of the outer sheath 100. The inner sleeve 200 can be locked to the outer sheath 100 when the clips 210 are fitted into receiving structures at the proximal end of the outer sheath 100. An example of a structure on the underside of the clips 210 is illustrated as the projection 211 of FIG. 3A. An example of a receiving structure at the proximal end of the outer sheath is illustrated as the cavity 120 in FIG. 1. In one embodiment, the end of the projection can form a snap-fit joint with the cavity in the outer sheath. The end can be deformable or flexible, wherein a force can be applied to fit the end of the projection into the opening of the cavity. The force can be applied, for example, to the clips 210. The force can cause the end of the projection to slightly deform or distort in order to fit through the opening of the cavity. In one embodiment, the opening of the cavity can have approximately the same dimensions as the end of the projection. In this manner, the inner sleeve 200 can be coupled to the outer sheath 100 until a force is applied to remove the projection from the cavity. Without the applied force of deformation, the end of the projection remains coupled to the cavity. The inner sleeve 200 can thus remain affixed to the outer sheath 100 and does not move independently of the outer sheath 100 until a force is applied to uncouple the inner and outer sheaths.

In one embodiment, the insertion of the inner sleeve 200 into the outer sheath 100 can result in the flexing of the vertical slats 115 of the outer sheath 100 and the expansion of the chamber formed by the outer sheath 10. The distal end and the outer wall of the inner sleeve 200 can push against the inner walls of the vertical slats 115 until the vertical slats 115 are straight rather than curved inward. When the vertical slats 115 are straightened, the flanges 110 at the distal ends of the slats can also extend outwards (away) from the central axis of the outer sheath. The flexing of the vertical slats 115 with the flanges 110 can result in a widening of the distal opening of the outer sheath such that the distal end of the outer sheath is wider than the incision. The extended flanges 110 can prevent movement of the outer sheath 100 in the incision, especially upward vertical movement of the outer sheath 100 out of the body. In one embodiment, the extended flanges 110 can be in contact with the inner wall of the thoracic cavity. The outer sheath 100 can thus be fixed in place by the extended flanges 110 when the inner sleeve 200 is fully inserted into the outer sheath 100. In some embodiments, the outer sheath 100 can remain in place during insufflation and can withstand pressures within and outside of the thoracic cavity in the expanded state.

In one embodiment, the inner sleeve 200 can be removed from the outer sheath 100 by applying a force to uncouple the inner sleeve 200 from the outer sheath 100. In one embodiment, the force can be applied to the inner sleeve 200. For example, a force can be applied to the clips 210 of the inner sleeve 200. The force can be a compressive (inward) force to push the clips 210 closer to each other. The inward force can cause the projection extending downwards from the inner sleeve to deform or curve and can result in the uncoupling of the projection from the cavity. In one embodiment, a combination of forces can be applied to the inner sleeve 200 and the outer sheath 100 to uncouple the inner sleeve 200 from the outer sheath 100. For example, a pushing force can be applied to the wings 120 of the outer sheath. The pushing force on the wings can distort the cavity in the outer sheath, e.g., widen the cavity, so that the projections of the inner sleeve can be released and removed from the cavity.

The inner sleeve 200 can be removed from the outer sheath 100 after being uncoupled from the outer sheath 100. In one embodiment, the removal of the inner sleeve 200 can result in the vertical slats 115 of the outer sheath 100 returning to their curved, tapered position. The outer sheath 100 can thus return to its retracted state, wherein the distal end of the sheath is narrower than the body of the sheath. The outer sheath 100 can be more easily removed from the body in the retracted state without causing irritation or trauma to the internal wall or the area surrounding the incision. In one embodiment, the outer sheath 100 can be removed from the incision by pulling on the wings 120 of the outer sheath. The wings 120 can provide a grip for handling the outer sheath 100 without contacting or blocking the chamber formed by the outer sheath 100.

FIG. 6B is a front view of the thoracic port 1000 in an expanded state, according to one embodiment of the present disclosure. In the expanded state, the inner sleeve 200 can sit flush against the outer sheath 100 such that the inner and outer sheaths can be fixed or maneuvered as a single component. For example, a rotation of the outer sheath in the expanded state can result in the same rotation of the inner sleeve. The distal flanges 110 of the outer sheath 100 can be approximately perpendicular to the central axis of the outer sheath 100 in the expanded state. In some embodiments, the top surfaces of the flange 110 can be in contact with the inner wall of the thoracic cavity when the thoracic port 1000 is in the expanded state. The contact can prevent removal of the thoracic port from the cavity in the expanded state.

FIG. 6C is a lateral view of the thoracic port 1000 in an expanded state, according to one embodiment of the present disclosure. The tether 305 can extend outwards from the body of the thoracic port. The tether 305 can be approximately in line with the proximal end of the outer sheath 100 and can lay flat against the skin when the port is inserted into the body.

FIG. 6D is an overhead view of the thoracic port 1000 in an expanded state, according to one embodiment of the present disclosure. The angled working channels 310, 320 of the cannulated core can be accessed from the proximal end of the thoracic port and can be extend to the distal opening of the thoracic port without being obscured by the distal flanges.

FIG. 6E is a view of the thoracic port 1000 in an expanded state from the distal end of the thoracic port, according to one embodiment of the present disclosure. The distal flanges 110 of the outer sheath are flared outwards such that the distal opening of the outer sheath is approximately a circle. The angled working channels 310, 320 of the cannulated core 300 can reach the distal opening of the port and are not blocked by the outer sheath. Instruments and/or devices can be inserted through the working channels and into the thoracic cavity when the thoracic port is in an expanded state. The working channels can be angled to prevent interference between inserted devices or tools.

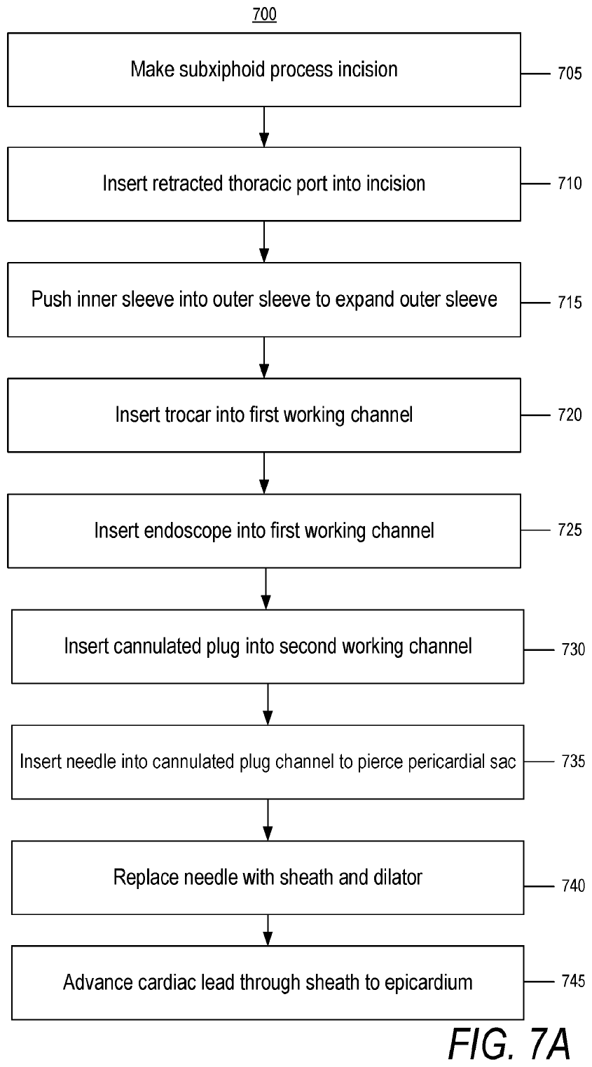

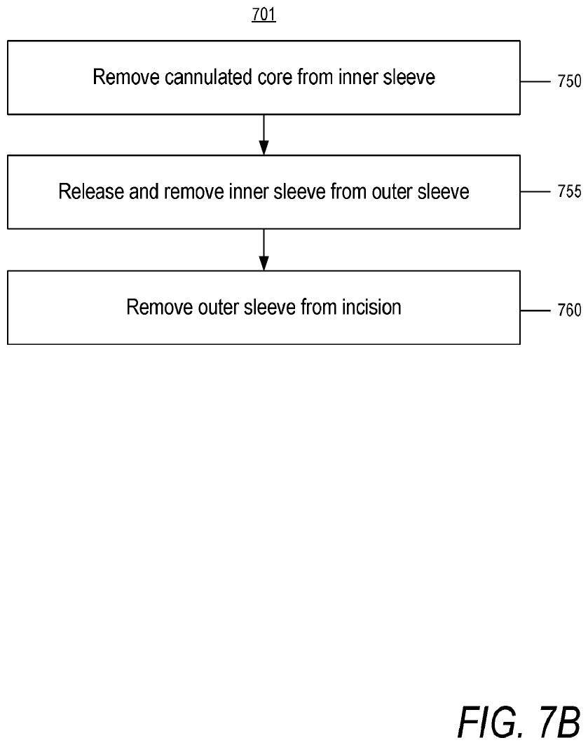

FIG. 7A is a method 700 for usage of the thoracic port 1000 to implant a lead on the epicardium, according to one embodiment of the present disclosure. The incision can first be made below the patient's sternum in step 705. The location of the incision can be determined by measuring a distance from the xiphoid process, wherein the distance is indicated by a reference feature on the tether of the thoracic port. The thoracic port can be assembled by fully inserting the cannulated core in the inner sleeve and partially inserting the inner sleeve in the outer sheath so that the outer sheath remains in a retracted state with a tapered distal end. The thoracic port can be inserted into the incision in the retracted state so that the tether with the cannulated plug extends towards the patient's head in step 710. The thoracic port can be expanded and locked in place in step 715 by pushing the inner sleeve so that it is fully inserted into and coupled to the outer sheath, causing the expansion of the outer sheath and the deployment of the distal flanges to be in contact with the inner wall of the thoracic cavity.

In step 720, a trocar can be inserted into the first working channel (e.g., the larger working channel) of the cannulated core to insufflate the thoracic cavity. In step 725, an endoscope can be passed through the trocar and maneuvered to provide visualization of the heart. The cannulated plug can be inserted into the second working channel (e.g., the smaller working channel) of the cannulated core to reduce the diameter of the working channel and limit leakage of insufflation gas in step 730. The cannulated plug can be inserted by folding the tether. In step 735, a needle can be passed through the working channel of the cannulated plug and into the thoracic cavity to pierce the pericardial sac. In step 740, the needle can be removed from the working channel and a sheath with a dilator can be inserted into the working channel and advanced into the pericardial space. In some implementations, the sheath with the dilator can be inserted into the pericardial space over a guide wire that has been inserted into the working channel. In step 745, one or more cardiac leads can be inserted through the sheath and fixed to the epicardium. The endoscope in the first working channel can provide a continuous view of the heart and the surrounding space throughout the steps of the method 700 so that the operator can properly advance the instruments through the second working channel and to the heart.

FIG. 7B is a method 701 for disassembly and removal of the thoracic port after implantation of a lead on the epicardium, according to one embodiment of the present disclosure. In step 750, the cannulated core can be removed from the inner sleeve. In one embodiment, the cannulated core can be removed by pulling the tether at or around the attachment of the tether to the core. In one embodiment, the cannulated core can be cut (e.g.,) by a scalpel in order to separate the core from the cardiac leads. The elastic material of the cannulated core can enable the cutting open of the core for disassembly. The cardiac leads can remain in place while the cannulated core is removed. The inner sleeve can be removed in step 755 by squeezing the clips of the inner sleeve to release the inner sleeve from the outer sheath and pulling the inner sleeve out from the outer sheath. The removal of the inner sleeve causes the distal flanges of the outer sheath to retract to form the tapered point at the distal end. The outer sheath can then be removed through the incision in step 760. The inner sleeve and the outer sheath can be threaded over the cardiac leads when they are removed from the body so that the cardiac leads remain in place. In some use cases, the cardiac leads can be held in place by one of the user's hands, while the user's other hand uncouples and removes the inner and outer sheaths. Advantageously, the method of assembly and insertion of the thoracic port, as well as disassembly and removal of the thoracic port can be executed by a single user. In addition, the various surgical instruments can be easily inserted into the thoracic cavity and maneuvered by a single operator without interference between the inserted instruments due to the angled working channels of the thoracic port.

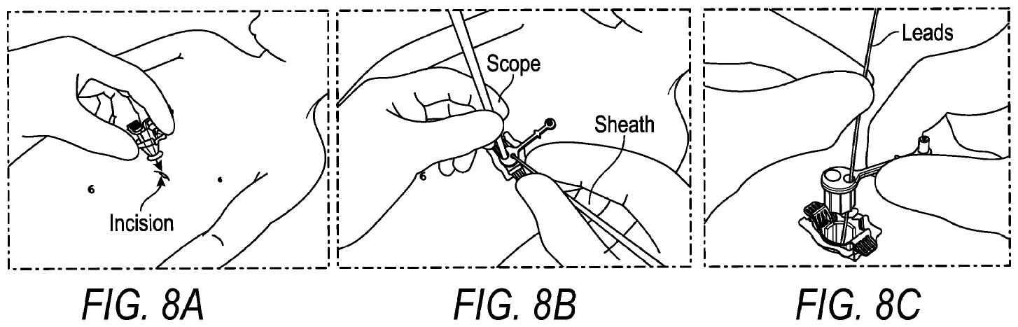

FIG. 8A through FIG. 8C are illustrations of usage of the thoracic port, according to one embodiment of the present disclosure. In FIG. 8A, the assembled thoracic port can be inserted into an incision in a retracted state. The inner sleeve can then be pushed into the outer sheath to engage the distal flanges and fix the thoracic port in the incision in an expanded state. In FIG. 8B, a scope can be advanced through a first working channel to provide a visualization of the thoracic cavity and relevant organs. A sheath can be advanced through a second working channel to access the desired cardiac region. The cannulated plug of the thoracic port, which extends via a tether towards the patient's head in FIG. 8B, may not be used for certain procedures using larger tools. In FIG. 8C, the leads can be implanted on the heart and the thoracic port can be disassembled by first removing the cannulated core 300, followed by the inner sleeve and the outer sheath. The cannulated core can be handled and maneuvered by moving the tether attached to the core.

The structures of the thoracic port described herein can provide many advantages and eliminate common failure points in delivering cardiac therapies. In some embodiments, the performance of the thoracic port under typical clinical forces (e.g., insertion force, deployment force, operating forces, drag forces, torque) can be validated using a combination of finite element analysis (FEA) and mechanical testing. For example, the retention of the thoracic port during usage can be tested to ensure that the thoracic port is not dislodged from the incision during regular operations. The thoracic port can be fixed to the body after insertion via the expansion of the outer sheath and the contact between the distal flanges and the inner wall of the thoracic cavity. The material composition of the thoracic port, including the exemplary materials listed herein, and the distal flanges of the thoracic port can withstand typical lateral and torsion forces and be retained in the incision. In some embodiments, portions of the thoracic port can be ductile and can bend or flex under stress without breaking or otherwise failing. For example, the distal flanges of the outer sheaths can be deformable to allow the thoracic port to be twisted while inserted into the body and in the expanded state. The twisting force can bend the flanges or cause the flanges to slip rather than shear. For example, 90° rotations applied to the thoracic port can result in a torque with a maximum magnitude of approximately 0.007±0.001 Nm (Newton-meters) and no fracture.

The thoracic port according to certain embodiments can withstand an insertion stress of approximately 2.21 MPa with a minimum factor of safety (FOS) of 6.78. A downward axial force of 10 N within the outer sheath can generate a maximum stress of approximately 11.49 MPa and a minimum FOS of 1.31, indicating that the thoracic port can withstand a force applied to expand the outer sheath and couple the inner sleeve to the outer sheath. A combination of upward (e.g., 10 N) and downward (e.g., 1 N) force applied to the thoracic port to simulate attempted dislodgment of the thoracic port can result in a maximum stress of approximately 6.34 MPa and a minimum FOS of 2.37. In some embodiments, the thoracic port can withstand an attempted dislodgment force of approximately 22 N. In some embodiments, the thoracic port can withstand torque of approximately 0.012 Nm, which is greater than typical torque forces applied during operation. For example, an applied torque of 0.01 Nm can generate a maximum stress of approximately 12.32 MPa with a minimum FOS of 1.32.

In some embodiments, the forces required to insert, couple, and otherwise use the components of the thoracic port can be measured to ensure that the necessary forces are within an expected range that could be manually applied in a clinical setting. In some implementations, the material properties of the thoracic port can affect the applied forces needed to operate the port. For example, the materials of the cannulated core and the inner sleeve can create a friction force between the cannulated core and the inner sleeve that can be overcome by an average user (e.g., a clinician) inserting the core into the inner sleeve. In one embodiment, the thoracic port can be operated under lubrication and sterilization conditions typically used in surgeries. In some embodiments, the thoracic port can slip and deform in order to withstand fracturing even when excess force is applied, minimizing risk of component breakage within the body.

According to some examples, a maximum force needed to push the thoracic port through skin can be approximately 0.78±0.19 N. A force applied overcome sliding friction of the inner sleeve against the outer sheath can be approximately 3.86±0.80 N. A force applied to lock the inner sleeve to the outer sheath and deploy the flanges in an expanded state can be approximately 9.06±1.55 N. In some embodiments, an average peak force of 11.51±1.85 N can be used to remove the thoracic port from an incision without disengaging the inner sleeve.

In typical procedures, multiple operators are needed to control a trocar, a scope, and any implantation instruments. The trocar and the scope must be constantly monitored and maneuvered to prevent the devices from interfering with the working path(s) of implantation instruments. The angled working channels of the surgical access port as described herein can reduce interference between inserted instruments and reduce the need for additional user assistance in accessing and visualizing the thoracic cavity. In one embodiment, the visualization channel can be designed to minimize slippage or lateral movement of an inserted instrument, while the second working channel can be designed to allow an inserted instrument to slide easily through the channel. For example, the dimensions and materials of the visualization channel can result in a drag force that minimizes unwanted movement, especially vertical movement, of a trocar and endoscope. In one implementation, the visualization channel can generate a drag force of approximately 3.97±1.65 N on an inserted tool, which represents a force needed to overcome static friction for insertion of the tool. The general sliding friction of the tool in the visualization channel can be approximately 0.91±0.48 N. The second working channel can generate a drag force of approximately 0.23±0.03 N on an inserted tool for easier movement within the second working channel. It can be appreciated that any of the foregoing measurements and quantities are included as exemplary features rather than as limitations or requirements for the design of the surgical access port presented herein.

The first working channel can be angled away from the second working channel such that the path of the trocar and the endoscope does not interfere with the path of the instruments advanced through the second working channel for implantation. In one embodiment, the angle of the working channels can direct the inserted devices and instruments towards target areas or structures in the thoracic cavity. For example, the second working channel can be angled towards the heart when the surgical access port is inserted into the thoracic cavity such that an inserted sheath can naturally be advanced towards the heart without being bent or kinked. The angled working channels can enable direct visualization while minimizing tool clashing and risk of heart perforation or damage to coronary vasculature. In some implementations, the surgical access port was determined to decrease physical demand, temporal demand, and frustration of cardiac therapy delivery.

The surgical access port of the present disclosure can provide a number of working channels of varying dimensions for use in different procedures requiring access to the thoracic cavity. According to one example, the thoracic port can include a first working channel and a second working channel in the cannulated core, as has been described herein. A cannulated plug can be inserted into the second working channel to further provide a third working channel for needles or similarly narrow instruments. In some embodiments, the cannulated core can be removed from the inner sleeve such that the chamber formed by the inner sleeve can be a large working channel or window to the thoracic cavity. Each of these working channels can be utilized in a procedure by inserting or removing the necessary components of a single thoracic port. In addition, the establishment of one working channel (e.g., the narrow working channel of the cannulated plug) can be reversed (e.g., by removing the cannulated plug from the working channel of the cannulated core) for further use. The flexible outer sheath of the thoracic port and the distal flanges can secure the thoracic port in place as the working channels are modified and used.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Embodiments of the present disclosure may also be as set forth in the following parentheticals:
(1) A surgical access port, comprising an outer sheath having an outer sheath having a tapered distal end; an inner sleeve fitting inside the outer sheath; and a cannulated core fitting inside the inner sleeve, wherein a distal opening of the outer sheath is configured to expand when the inner sleeve is inserted into the outer sheath, and wherein the cannulated core forms a first working channel and a second working channel between a first end of the cannulated core and an opposing second end of the cannulated core.
(2) The surgical access port of (1), wherein the outer sheath forms a flange surrounding the distal opening of the outer sheath.
(3) The surgical access port of (1) to (2), wherein a proximal end of the inner sleeve is removably coupled to a proximal end of the outer sheath.
(4) The surgical access port of (1) to (3), wherein the proximal end of the inner sleeve includes a deformable projection configured to fit into a cavity in the proximal end of the outer sheath.
(5) The surgical access port of (1) to (4), wherein the cannulated core is an elastic material.
(6) The surgical access port of (1) to (5), wherein the second working channel is narrower than the first working channel.
(7) The surgical access port of (1) to (6), further comprising a cannulated plug connected to the cannulated core via a tether, the cannulated plug forming a third working channel, wherein the cannulated plug is configured to be inserted into the second working channel and wherein the third working channel is narrower than the second working channel.
(8) A surgical access port, comprising an outer sheath having a tapered distal end; an inner sleeve fitting inside the outer sheath; and a cannulated core fitting inside the inner sleeve, wherein a distal opening of the outer sheath is configured to expand when the inner sleeve is inserted into the outer sheath, wherein the cannulated core forms a first working channel and a second working channel between a first end of the cannulated core and an opposing second end of the cannulated core, and wherein the first working channel and the second working channel form an angle of separation.
(9) The surgical access port of (8), wherein the outer sheath forms a flange surrounding the distal opening of the outer sheath.
(10) The surgical access port of (8) to (9), wherein a proximal end of the inner sleeve is removably coupled to a proximal end of the outer sheath.
(11) The surgical access port of (8) to (10), wherein the proximal end of the inner sleeve includes a deformable projection configured to fit into a cavity in the proximal end of the outer sheath.
(12) The surgical access port of (8) to (11), wherein the second working channel is narrower than the first working channel.
(13) The surgical access port of (8) to (12), wherein the angle of separation between the first working channel and the second working channel is an acute angle.
(14) The surgical access port of (8) to (13), further comprising a cannulated plug connected to the cannulated core via a tether, the cannulated plug forming a third working channel, wherein the cannulated plug is configured to be inserted into the second working channel and wherein the third working channel is narrower than the second working channel.
(15) A surgical access port, comprising an outer sheath having a tapered distal end; an inner sleeve fitting inside the outer sheath; and a cannulated core fitting inside the inner sleeve, wherein a distal opening of the outer sheath is configured to expand when the inner sleeve is inserted into the outer sheath, wherein the inner sleeve is removably coupled to the outer sheath, wherein the cannulated core forms a first working channel and a second working channel between a first end of the cannulated core and an opposing second end of the cannulated core, wherein the first working channel and the second working channel form an angle of separation, and wherein an outer wall of the cannulated core forms a notch at a proximal end of the cannulated core oriented towards a proximal opening of the second working channel.
(16) The surgical access port of (15), wherein the outer sheath forms a flange surrounding the distal opening of the outer sheath.
(17) The surgical access port of (15) to (16), wherein the inner sleeve includes a deformable projection configured to fit into a cavity in the outer sheath.
(18) The surgical access port of (15) to (17), wherein the angle of separation between the first working channel and the second working channel is an acute angle.
(19) The surgical access port of (15) to (18), wherein the second working channel is narrower than the first working channel.
(20) The surgical access port of (15) to (19), further comprising a cannulated plug connected to the cannulated core via a tether, the cannulated plug forming a third working channel, wherein the cannulated plug is configured to be inserted into the second working channel and wherein the third working channel is narrower than the second working channel.
(21) The surgical access port of (1) to (7), wherein the tether includes a demarcation along a length of the tether.
(22) The surgical access port of (8) to (14), wherein the tether includes a demarcation along a length of the tether.
(23) The surgical access port of (15) to (20), wherein the tether includes a demarcation along a length of the tether.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A surgical access port, comprising:
an outer sheath having a tapered distal end;
an inner sleeve fitting inside the outer sheath; and
a cannulated core fitting inside the inner sleeve,
wherein a distal opening of the outer sheath is configured to expand when the inner sleeve is inserted into the outer sheath, and
wherein the cannulated core forms a first working channel and a second working channel between a first end of the cannulated core and an opposing second end of the cannulated core.

2. The surgical access port of claim 1, wherein the outer sheath forms a flange surrounding the distal opening of the outer sheath.

3. The surgical access port of claim 1, wherein a proximal end of the inner sleeve is removably coupled to a proximal end of the outer sheath.

4. The surgical access port of claim 3, wherein the proximal end of the inner sleeve includes a deformable projection configured to fit into a cavity in the proximal end of the outer sheath.

5. The surgical access port of claim 1, wherein the cannulated core is an elastic material.

6. The surgical access port of claim 1, wherein the second working channel is narrower than the first working channel.

7. The surgical access port of claim 1, further comprising a cannulated plug connected to the cannulated core via a tether, the cannulated plug forming a third working channel, wherein the cannulated plug is configured to be inserted into the second working channel and wherein the third working channel is narrower than the second working channel.

8. The surgical access port of claim 7, wherein the tether includes a demarcation along a length of the tether.

9. A surgical access port, comprising:
an outer sheath having a tapered distal end;
an inner sleeve fitting inside the outer sheath; and
a cannulated core fitting inside the inner sleeve,
wherein a distal opening of the outer sheath is configured to expand when the inner sleeve is inserted into the outer sheath,
wherein the cannulated core forms a first working channel and a second working channel between a first end of the cannulated core and an opposing second end of the cannulated core, and
wherein the first working channel and the second working channel form an angle of separation.

10. The surgical access port of claim 9, wherein the outer sheath forms a flange surrounding the distal opening of the outer sheath.

11. The surgical access port of claim 9, wherein a proximal end of the inner sleeve is removably coupled to a proximal end of the outer sheath.

12. The surgical access port of claim 11, wherein the proximal end of the inner sleeve includes a deformable projection configured to fit into a cavity in the proximal end of the outer sheath.

13. The surgical access port of claim 9, wherein the second working channel is narrower than the first working channel.

14. The surgical access port of claim 9, wherein the angle of separation between the first working channel and the second working channel is an acute angle.

15. The surgical access port of claim 9, further comprising a cannulated plug connected to the cannulated core via a tether, the cannulated plug forming a third working channel, wherein the cannulated plug is configured to be inserted into the second working channel and wherein the third working channel is narrower than the second working channel.

16. A surgical access port, comprising:
an outer sheath having a tapered distal end;
an inner sleeve fitting inside the outer sheath; and
a cannulated core fitting inside the inner sleeve,
wherein a distal opening of the outer sheath is configured to expand when the inner sleeve is inserted into the outer sheath,
wherein the inner sleeve is removably coupled to the outer sheath,
wherein the cannulated core forms a first working channel and a second working channel between a first end of the cannulated core and an opposing second end of the cannulated core,
wherein the first working channel and the second working channel form an angle of separation, and
wherein an outer wall of the cannulated core forms a notch at a proximal end of the cannulated core oriented towards a proximal opening of the second working channel.

17. The surgical access port of claim 16, wherein the outer sheath forms a flange surrounding the distal opening of the outer sheath.

18. The surgical access port of claim 16, wherein the angle of separation between the first working channel and the second working channel is an acute angle.

19. The surgical access port of claim 16, wherein the second working channel is narrower than the first working channel.

20. The surgical access port of claim 16, further comprising a cannulated plug connected to the cannulated core via a tether, the cannulated plug forming a third working channel, wherein the cannulated plug is configured to be inserted into the second working channel and wherein the third working channel is narrower than the second working channel.
